(19) United States
(12) Patent Application Publication
(10) Pub. No.: US 2016/0367218 A1
(43) Pub. Date: Dec. 22, 2016

(54) APPARATUS AND METHOD FOR MEASURING AN AMOUNT OF URINE IN A BLADDER

(71) Applicant: Mcube Technology Co., Ltd., Seoul (KR)

(72) Inventors: Jung-Hoe KIM, Seoul (KR); Seung-Tai KIM, Gwacheon-si (KR)

(21) Appl. No.: 15/252,659

(22) Filed: Aug. 31, 2016

Related U.S. Application Data
(63) Continuation-in-part of application No. 15/072,687, filed on Mar. 17, 2016.
Application No. 15/072,687 is a continuation-in-part of application No. 13/029,798, filed on Feb. 17, 2011.
Application No. 13/029,798 is a continuation-in-part of application No. 12/045,680, filed on Mar. 10, 2008, now abandoned.

(51) Int. Cl. A61B 8/00; A61B 8/08

(52) U.S. Cl. CPC A61B 8/4461; A61B 8/5207

(57) ABSTRACT

An apparatus for measuring an amount of urine in a bladder includes a transducer, a switch for selecting one of operational modes, which include a bladder position check mode and a urine amount measurement mode, and a central control unit for operating the apparatus according to the operational mode selected by the switch. The central control unit in the bladder position check mode repeats sequentially predetermined operations until the urine amount measurement mode is selected, the predetermined operations include performing a preliminary scan to a single scan plane related to an aiming direction of the transducer, detecting a center point of the bladder in a ultrasound image, and representing a predetermined mark at the center point of the bladder in the displayed ultrasound image in order to indicate visually a center point of the bladder.

This application is a continuation-in-part of U.S. Ser. No. 15/072,687, filed on Mar. 17, 2016, which is a continuation-in-part of U.S. Ser. No. 13/029,798, filed on Feb. 17, 2011, which is a continuation-in-part of U.S. Ser. No. 12/045,680, filed on Mar. 10, 2008.

### BACKGROUND OF THE INVENTION

1. Field of the Invention

The present invention relates generally to an apparatus for measuring an amount of urine in a bladder using ultrasound signals and, more particularly, to an apparatus which detects the exact location of a bladder with respect to an aiming direction of a transducer and then performs the ultrasound scan for measuring an amount of urine in order to measure quickly and accurately the amount of urine in the urinary bladder.

2. Description of the Prior Art

Generally, an ultrasound system is a system that emits ultrasound signals to an object to be examined using the piezoelectric effect of a transducer, receives the ultrasound signals reflected from the discontinuous planes of the object, converts the received ultrasound signals into electrical signals, and outputs the electrical signals to a predetermined display device, thus enabling examination of the internal state of the object. Such an ultrasound system is widely used for medical diagnosis equipment, non-destructive testing equipment and underwater detection equipment.

However, most conventional ultrasound diagnosis apparatuses are inconvenient in that they cannot be easily carried due to their large size and heavy weight. To solve the inconvenience, various portable ultrasound diagnosis apparatuses have been proposed. Korean Utility Model Registration No. 20-137995 discloses a “Portable ultrasound Diagnosis Apparatus”.

Meanwhile, when examining bladder abnormalities or urinary difficulty, measuring the amount of urine is an essential procedure. Furthermore, prior to urination using a catheter, the amount of urine in the urinary bladder should be measured to account for urine that may be retained after the operation. In addition, in urination training, the amount of urine in the urinary bladder should be measured as a guideline.

Various types of ultrasound scanning equipment may be used to measure the amount of urine in the urinary bladder, as described above. In this case, two methods are used. A first method calculates the amount of urine from respective ultrasound images for a perpendicular plane and a horizontal plane, which are obtained using typical ultrasound scanning equipment.

However, although many algorithms has been proposed and used for the method, the first method is problematic in that it not only exhibits a considerable error rate but also exhibits different results for different users.

A second method uses dedicated ultrasound equipment for measuring the amount of urine. U.S. Pat. No. 4,926,871 and U.S. Pat. No. 6,884,217 disclose dedicated ultrasound equipments. However, the dedicated ultrasound equipments based on the second method have a disadvantage in that they also calculate the amount of urine chiefly using two ultrasound images, which are related to the perpendicular and horizontal planes of the urinary bladder, respectively, and in that a user must find the area indicating the greatest size and select it in order to calculate the amount of urine.

Accordingly, the apparatus and method disclosed herein are designed to overcome the above disadvantages of conventional equipments.

### SUMMARY OF THE INVENTION

Accordingly, the present invention is an apparatus for measuring an amount of urine in a bladder using ultrasound signals, comprising: a transducer for transmitting ultrasound signals and receiving ultrasound signals reflected to and from the bladder and tissues surrounding the bladder; a switch for selecting one of operational modes, which include a bladder position check mode and a urine amount measurement mode; a display unit for outputting images; and a central control unit for operating the apparatus according to the operational mode selected by the switch,

wherein the central control unit in the bladder position check mode is configured to repeat sequentially predetermined operations until the urine amount measurement mode is selected, the predetermined operations include to perform a preliminary scan to obtain ultrasound signals for a plurality of scan lines of a single scan plane related to an aiming direction of the transducer, to detect location information of a bladder in a ultrasound image by using the obtained ultrasound signals of the single scan plane, to detect a center point of the bladder by using the location information of the bladder, and to represent a predetermined mark at the center point of the bladder in the displayed ultrasound image in order to indicate visually a center of the bladder,

wherein the central control unit in the urine amount measurement mode is configured to perform an main scan to obtain ultrasound signals for a plurality of scan planes from the transducer, to estimate a volume of the bladder by using the ultrasound signals obtained by the main scan, and to measure an amount of urine corresponding to the estimated volume of the bladder.

In the apparatus for measuring an amount of urine, it is preferred that the central control unit be configured to, in the bladder position check mode, mark a vertical center line on the center of the ultrasound image.

In addition, it is preferred that the predetermined mark be one of a cross mark, an arrow and a geometrical figure on the center point.

The apparatus according to the present invention provides visually the information of the central location of the urinary bladder on a display in the bladder position check mode that a user desires to examine and then measures the amount of urine at the location which is selected by user according to the provided information of the central location of the urinary bladder in the urine amount measurement mode. As a result, the amount of urine in the urinary bladder can also be quickly and accurately measured.

### BRIEF DESCRIPTION OF THE DRAWINGS

FIG. 1 is a block diagram schematically showing the internal construction of the apparatus according to the preferred embodiment of the present invention.

FIG. 2 is a perspective view showing the apparatus according to the preferred embodiment of the present invention.

FIG. 3A is a conceptual diagram illustrating of acquiring the 2-dimensional ultrasound image for a single scan plane using the apparatus according to the present invention.

FIG. 3B is an example of the 2-dimensional B-mode ultrasonic image generated in the preliminary scan mode according to the present invention.

FIG. 4 is a flowchart sequentially illustrating processes of obtaining the amount of the urine in the urinary bladder by the central control unit in the apparatus according to the present invention.

FIGS. 5A and 5B are diagrams illustrating screens of the display unit displaying an ultrasound image represented a center point of a bladder according to the first implementation of the bladder position check mode in the apparatus according to the present invention.

FIGS. 6A and 6B are diagrams illustrating screens of the display unit displaying an ultrasound image represented a center point of a urinary bladder and a vertical center line of the ultrasound image according to the second implementation of the bladder position check mode in the apparatus according to the present invention.

FIG. 7 is a diagram illustrating the processes of detecting the center point of the bladder in the apparatus according to the present invention.

### DETAILED DESCRIPTION OF THE INVENTION

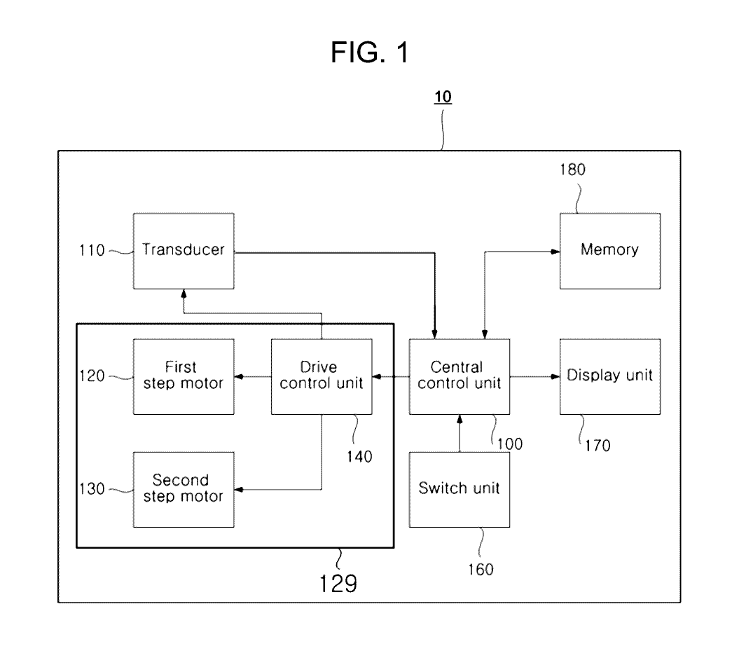

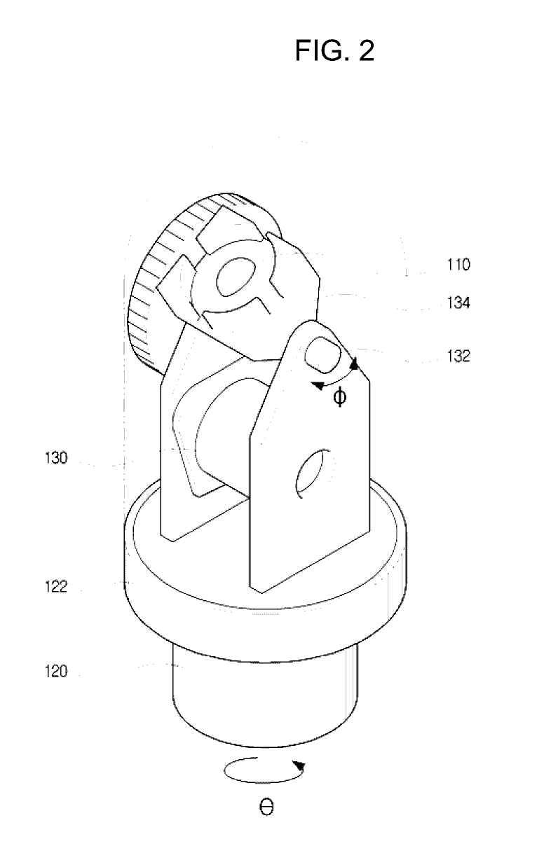

The construction and operation of an apparatus for measuring an amount of urine in a urinary bladder according to a preferred embodiment of the present invention are described in detail with reference to the accompanying drawings below. FIG. 1 is a block diagram schematically showing the internal construction of the apparatus according to the preferred embodiment of the present invention, and FIG. 2 is a perspective view showing the apparatus according to the preferred embodiment of the present invention.

Referring to FIG. 1, the apparatus 10 according to the preferred embodiment of the present invention includes a central control unit 100 for controlling the overall operation of the apparatus, a transducer 110, a first stepping motor 120, a second stepping motor 130, a drive control unit 140, an analog signal processing unit 150, a switch unit 160, a memory 180, and a display unit 170. The respective components of the above-described apparatus 10 are described in detail below.

The transducer 110 transmits ultrasound signals and receives ultrasound signals reflected from the internal organs of a human body, and transmits the received ultrasound signals to the central control unit 100 after converting to digital signals through the analog signal processing unit 150.

The analog signal processing unit 150 converts an analog signals input from the transducer 110 to a digital signals and transmits to the central control unit 100.

The switch 160 performs input to select operational modes, such as a bladder position check mode and a urine amount measurement mode. The switch 160 according to a preferred embodiment of the present invention enables an operational mode, depending on input time or input form, to be determined using a single switch. In addition, another embodiment of the switch 160 of the present invention may be configured to be provided with a plurality of buttons, and allow different buttons to be assigned to respective operational modes.

As shown in FIG. 2, a rotational support module 122 is fixed to the first stepping motor 120. A second stepping motor 130 is mounted on the rotational support module 122. The second stepping motor 130 is connected with a rotational axis 132, and a transducer support module 134 is fixed to the rotational axis 132. A transducer 110 is installed in the transducer support module 134.

The central control unit 100 generates and transmits drive control signals to the drive control unit 140 in response to a request signal received from the switch unit 160, and the drive control unit 140 controls the motion of the first and second stepping motors 120 and 130 in response to the drive control signals, so that the ultrasound signals for scan planes can be obtained through the rotation of the transducer 110.

The second stepping motor 130 moves the transducer 110 in the phi(Φ) dimension of a single scan plane. The angle between two boundary edges of the scan plane, that is a total angle phi(Φ), can vary, but typically will be approximately 120°. The rotational axis 132 and the transducer support module 134, which are connected to the second stepping motor 130 using a connection unit, are rotated by the second stepping motor 130. The connection unit can be used a gear, a belt or etc.

To acquire ultrasound signals for a plurality of scan planes, the first stepping motor 120 rotates the transducer assembly about a central vertical axis through a total angle theta(θ) in series of small angular movements. Typically, the total angle theta will be 360°. The first stepping motor rotates successively a predetermined angle in the theta direction, at each theta position a plurality of ultrasound signals for a scan plane are acquired according to the above-described process. The total number of scan planes over the entire theta dimension of 360° will completely sample the 3-dimensional ultrasound scan cone by ultrasound signals. The ultrasound signals are processed to determine the locations of the bladder walls in each of the scan planes.

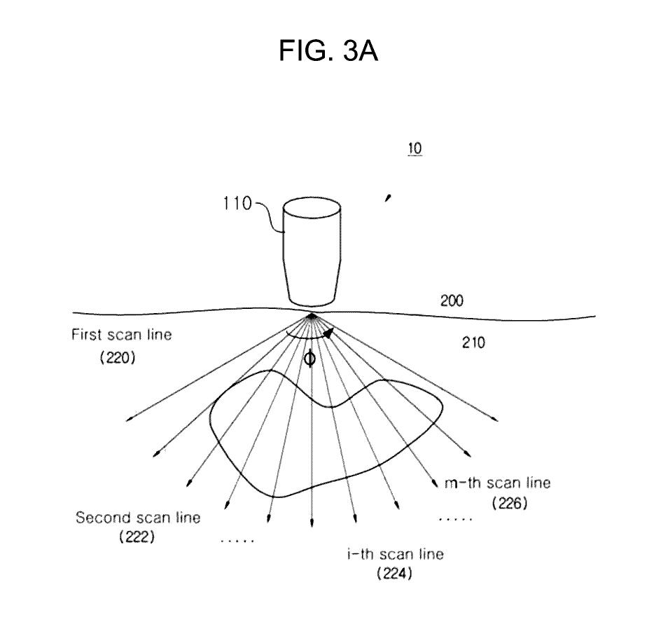

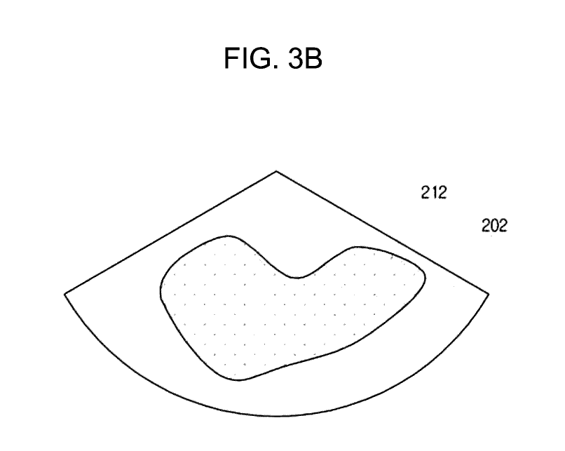

FIG. 3A is a diagram illustrating a process of the apparatus 10 according to the present invention, acquiring a bladder image for a single plane. FIG. 3B is an example of the 2-dimensional B-mode ultrasonic image generated in the preliminary scan mode according to the present invention.

With reference to FIG. 3A, in the apparatus 10 in which the transducer is disposed on an arbitrary location of an abdomen 200 over the urinary bladder 210 of a patient, the central control unit causes the first stepping motor and the second stepping motor to be fixed, and detects ultrasound signals at the corresponding location. Thereafter, a process of detecting ultrasound signals at a corresponding angle while moving the second stepping motor by the predetermined angle in the yz direction is repeated, and thus ultrasound signals for n scan lines, that is, a first scan line 220, a second scan line 222, . . . , a i-th scan line 224, . . . , a n-th scan line 226 are sequentially detected. After detecting n ultrasound signals, the central control unit 100, as shown in FIG. 3B, generates a two-dimensional image by processing ultrasound signals for a corresponding plane, and displays the generated two-dimensional image on the display unit 170. FIG. 3B is a diagram showing the two-dimensional image output to the display unit 170, in which urine 212 in the urinary bladder 210 is displayed while being separated from organs 202 around the urinary bladder 210.

Meanwhile, the above-described process is repeated while the first stepping motor is rotated by the predetermined angle and, thus, ultrasound signals for the n scan lines for the m planes are detected. As described above, a three-dimensional image is generated using a plurality of two-dimensional images acquired for the m planes. In this case, it is preferred that the number m of the acquired two-dimensional images be equal to or greater than 4 and equal to and less than 30.

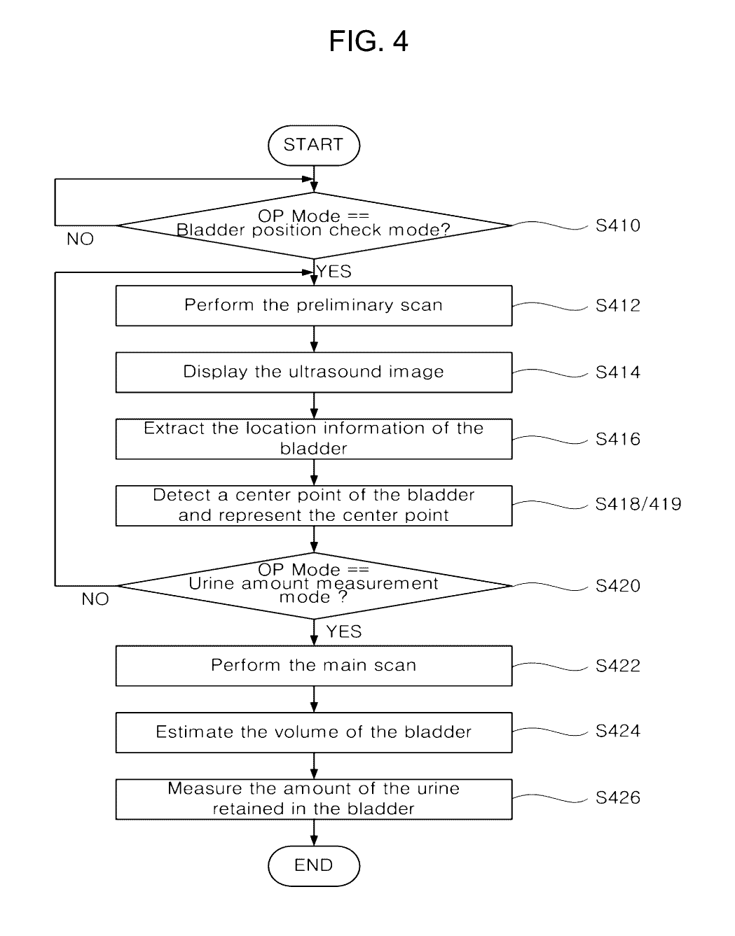

The central control unit 100 determines an operational mode based on a signal input through the switch 160. FIG. 4 is a flowchart sequentially illustrating processes of obtaining the amount of the urine amount in the bladder by the central control unit 100 of the apparatus according to the present invention.

Referring to FIG. 4, the central control unit 100 of the present invention determines an operational mode based on a signal input through the switch 160, at step 400. If the operational mode is the bladder position check mode, the central control unit 100 performs repeatedly and sequentially the predetermined operations until the urine amount measurement mode is selected. The predetermined operations in the bladder position check mode include to perform a preliminary scan to obtain ultrasound signals for a plurality of scan lines of a single scan plane from the transducer, at step 412, to generate and display an ultrasound image by using the obtained the ultrasound signals for a plurality of scan lines of a single scan plan, at step 414, to detect location information of a bladder in the ultrasound image using the obtained ultrasound signals to the single scan plane, at step 416, to detect a center point of the bladder by using the location information of the bladder, at step 418, and to represent a predetermined mark on the center point of the bladder in the displayed ultrasound image, at step 419, in order to indicate visually a center position of the bladder.

If the operational mode is the urine amount measurement mode, at step 420, the central control unit 100 performs a main scan to obtain ultrasound signals for a plurality of scan planes from the transducer, at step 422, to estimate a volume of the bladder by using the ultrasound signals obtained by the main scan, at step 424, and to measure an amount of urine corresponding to the estimated volume of the bladder, at step 426.

In this case, in the state in which the transducer is disposed on the abdomen of a patient and is oriented toward his or her urinary bladder in the bladder position check mode, the transducer of the apparatus according to the present invention rotates in left and right directions or in up and down directions relative to the patient, that is, a lateral direction with respect to the patient, and thus a two-dimensional B-mode ultrasound image obtained as a result of the rotation is output to the display unit.

In use, the operator of the apparatus checks the displayed ultrasound image in the bladder position check mode. If the center point of the bladder is located on a desirable position or corresponds with the vertical center line of the ultrasound image, then the operator may select the urine amount measurement mode to measure accurately the amount of the urine in the bladder at the optimal orientation direction of the transducer. If not, the operator should re-aim or re-orient the transducer of the apparatus for accurate measurement.

The operation of the central control unit according to the first implementation of the bladder position check mode of the invention is described below.

When the operational mode is the bladder position check mode, the central control unit 100 performs repeatedly and sequentially the predetermined operations until the urine amount measurement mode is selected in order to display a 2-dimensional ultrasound image which is fan-shaped and in which a predetermined mark is represented on a center point of a bladder. The predetermined mark may be one of a cross mark, an arrow and a geometrical figure.

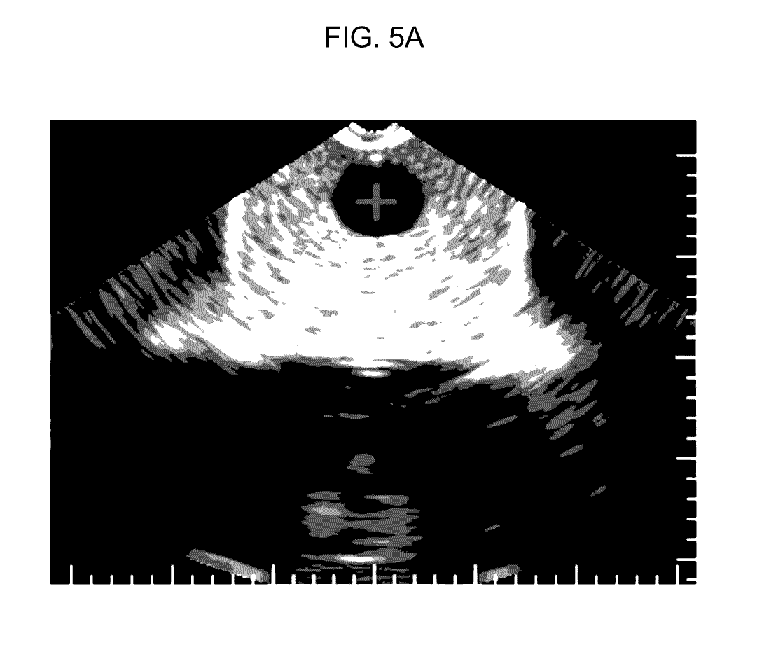

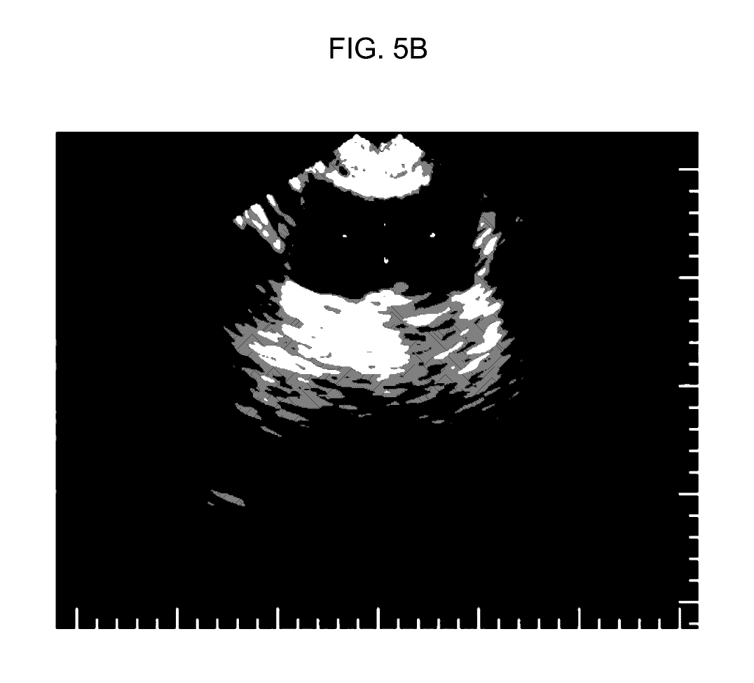

FIGS. 5A and 5B are diagrams illustrating screens of the display unit displaying an ultrasound image represented a center point of a bladder according to the first implementation of the bladder position check mode in the apparatus according to the present invention. Referring to the FIG. 5, a cross mark ‘+’ is represented on the center point of the bladder in the 2-dimensional ultrasound image.

Furthermore, in the bladder position check mode, the above-described process is periodically repeated until the urine amount measurement mode is selected and a two-dimensional ultrasound image represented the center point of the bladder for a corresponding plane is output to the display unit. In this case, it is preferred that the repetition period of the predetermined operation be less than about 5 seconds.

The predetermined operations in the bladder position check mode include to perform a preliminary scan to obtain ultrasound signals for a plurality of scan lines of a single scan plane from the transducer, at step 412, to generate and display an ultrasound image by using the obtained the ultrasound signals for a plurality of scan lines of a single scan plan, at step 414, to detect location information of a bladder in the ultrasound image using by the obtained ultrasound signals to the single scan plane, at step 416, to detect a center point of the bladder using by the location information of the bladder, at step 418, and to represent a predetermined mark on the center point of the bladder in the displayed ultrasound image, at step 419, in order to represent visually a center position of the bladder.

In the preliminary scan of the step 412, in the state in which the transducer is disposed on the patient's abdomen and is oriented toward his or her urinary bladder, the apparatus according to the present invention rotates in left and right directions relative to the patient, that is, a lateral direction with respect to the patient, and thus obtains the ultrasound signals of a plurality of scan lines to a single scan plane.

In this case, in order to obtain the ultrasound signals comprising of the single scan plane in the preliminary scan of the step 412, the central control unit transmits a drive control signal for sequentially rotating the second stepping motor to the drive control unit, and the drive control unit rotates the second stepping motor in a yz direction (that is, a second direction) in response to the drive control signal received from the central control unit. As the second stepping motor rotates, the transducer also rotates. The transducer acquires the pieces of ultrasound information of n scan lines in the yz direction while rotating in the yz direction. Meanwhile, the central control unit receives the pieces of ultrasound information of n scan lines in the yz direction from the transducer, generates a ultrasound image for a corresponding plane in the yz direction from the pieces of received ultrasound information, and outputs the generated ultrasound image to the display unit.

The ultrasound image is preferably a 2-dimensional B-mode ultrasound image.

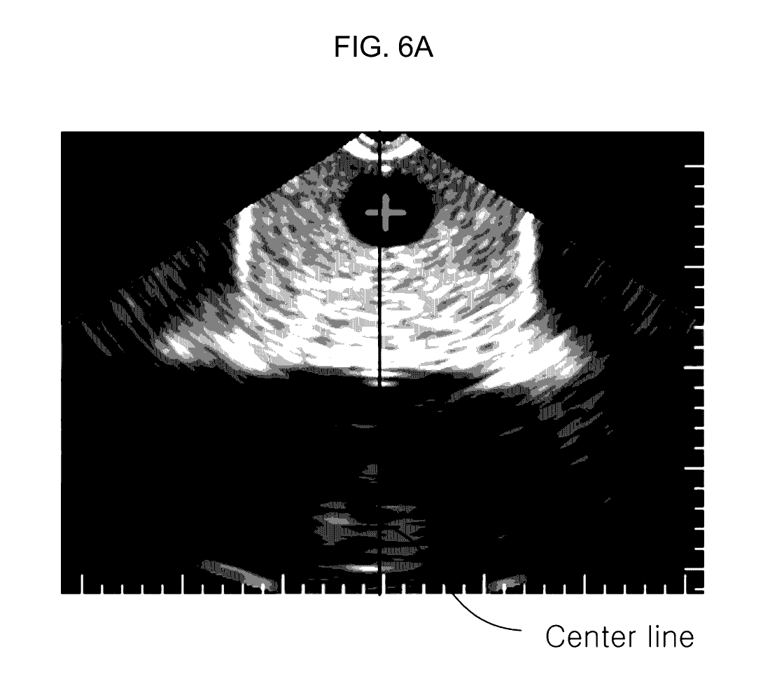

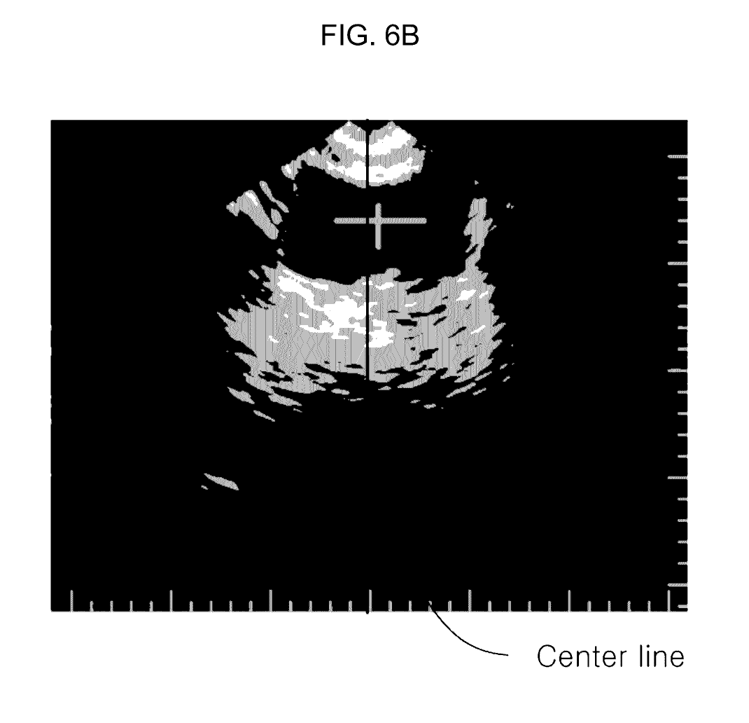

In the second implementation of the bladder position check mode in the apparatus according to the present invention, the predetermined operations of the bladder position check mode further include to mark a vertical center-line on the center of the ultrasound image. FIGS. 6A and 6B are diagrams illustrating screens of the display unit displaying an ultrasound image represented a center point of a urinary bladder and a vertical center line of the ultrasound image according to the second implementation of the bladder position check mode in the apparatus according to the present invention.

Referring to FIGS. 6A and 6B, if the center point of the urinary bladder matches the vertical center line of the ultrasound image, then the operator can determine visually and quickly the accuracy of the orientation of the transducer.

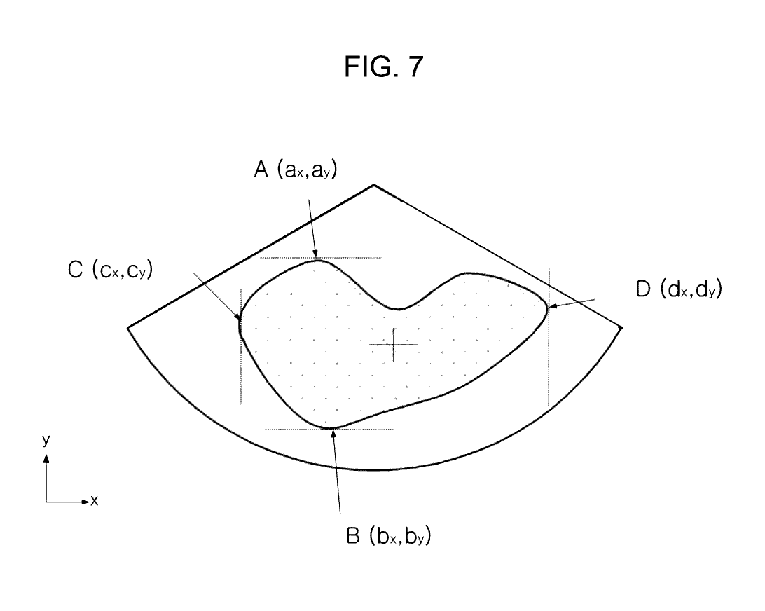

One example of a method to detect the center point of the bladder is described below. FIG. 7 is a diagrams illustrating one example of the processes of detecting the center point of the bladder in the apparatus according to the present invention. At first, the location information corresponded to the front and the back walls of the bladder are detected from a plurality of the ultrasound scan lines comprising the single scan plane which is obtained by the preliminary scan.

Referring to FIG. 7, the four points corresponding to the top and the bottom locations A and B and the left and the right locations C and D respectively are extracted from the detected location information corresponded to the front and the back walls of the bladder. In this example, the x, y coordinates of the top point A are aₓ and a_{y}, the x, y coordinates of the bottom point B are bₓ and b_{y}, the x, y coordinates of the most left point C are cₓ and c_{y}, and the x, y coordinates of the most right point D are dₓ and d_{y}. Then, the center point of the bladder can be set as the x, y coordinates, (cₓ+dₓ)/2 and (a_{y}+b_{y})/2), which the value (cₓ+dₓ)/2 is a median value of bladder with respect to the x-axis and the value (a_{y}+b_{y})/2 is a median value of the bladder with respect to the y-axis.

The central control unit, in the bladder position check mode, indicates the mark on the center point of the bladder by using one of a cross mark, an arrow and a geometrical figure in the ultrasound image.

In this embodiment, the central control unit according to the invention can measure the cross-sectional area of the bladder and determine the size of the mark indicating the center point of the bladder in proportion to the measured the cross-sectional area of the bladder. In one example, the apparatus sets initially the specific sizes of the mark according to the cross-sectional areas of the bladder and stores the sizes in the database. When the apparatus according to the invention displays the mark on the center point of the bladder in the ultrasound image, the central control unit reads the size of the mark corresponding to the cross-sectional area of the bladder from the database and indicates the mark with the read size at the center point of the bladder.

Referring to the FIGS. 5A and 5B, the cross-sectional area of the bladder of FIG. 5A is smaller than that of FIG. 5B, and thus the mark size of FIG. 5A is smaller than that of FIG. 5B. By changing the mark size in proportion to the cross-sectional areas of the bladder, the operator can understand visually the size of the bladder with respect to the scan plane and determine quickly the accuracy of the orientation of the transducer.

When the urine amount measurement mode is selected under the bladder position check mode, the main control unit is configured to (1) perform a main scan to obtain ultrasound signals for a plurality of scan planes from the transducer, at step 422, (2) estimate a volume of the bladder by using the ultrasound signals obtained by the main scan, at step 424, and (3) measure an amount of urine corresponding to the estimated volume of the bladder, at step 426.

The process of measuring the amount of the urine in the bladder in the urine amount measurement mode according to the present invention is described below.

The central control unit 100 receives ultrasound signals for a plurality of scan planes from the transducer, which each of the scan planes is separated by a selected angle and consists of a plurality of scan lines to produce a scan cone for scanning 3-dimensionally the bladder.

In this embodiment, the above-described process is repeated while the first stepping motor is rotated by the predetermined angle and, thus, ultrasound signals for the n scan lines for the m scan planes are detected. As described above, a three-dimensional image is generated using two-dimensional images acquired for the m planes. In this case, it is preferred that the number m of the acquired two-dimensional images be equal to or greater than 4 and equal to and less than 30.

Accordingly, the apparatus according to the invention can measure the three-dimensional volume of the bladder by using the ultrasound signals obtained by the movement of the first and second stepping motors.

A method of the central control unit 100 of the apparatus according to the preferred embodiment of the present invention, in the urine amount measurement mode, measuring the amount of urine in the urinary bladder using ultrasound signals, is described below.

The central control unit 100 of the apparatus 10 according to the invention obtains the ultrasound signals of a plurality of the scan lines with respect to the scan planes. Thereafter, the locations of front and back walls are detected from pieces of ultrasound information of scan lines constituting each plane, and distance values Depth[1], Depth[2], . . . , Depth[n] corresponding to the distances between the locations of the detected front and back walls for the respective scan lines are obtained. Thereafter, the area of the corresponding plane is obtained by summing the distance values for the scan lines constituting each plane.

The above-described process of obtaining the area of each plane is repeatedly performed on m planes, and thus the areas Area[1], Area[2], . . . , Area[m] of the respective planes are obtained. In this case, the method of obtaining the area of each plane using distance values corresponding to the distances between the locations of the front and back walls of the urinary bladder for the respective scan lines may be implemented in various ways. As an example, the entire area of each plane may be obtained by obtaining an area for a sector for a single scan line using the rotational angle of the second stepping motor 130 and summing sector areas for respective lines having back walls. As another example, the entire area may be obtained by summing trapezoidal areas, which are obtained by repeating a process of obtaining an area for a trapezoid, which is formed by the two front walls and two back walls of two neighboring scan lines.

Meanwhile, if scanning is performed in a state in which the center of a first rotational axis moves from the center of the urinary bladder when a three-dimensional volume is obtained using a plurality of two-dimensional images, an amount smaller than an actual amount is calculated and, thus, an error relative to the actual amount is generated. Accordingly, numerical correction is performed to reduce such error and accurately measure the amount of urine in the urinary bladder. The process of performing the numerical correction is described below.

First, the distance values corresponding to the distances between the locations of front and back walls of the urinary bladder for n scan lines constituting each plane are obtained. Thereafter, the maximum distance values bladderDepth[1], bladderDepth[2], . . . , bladderDepth[m] of the respective planes are obtained among the distance values, and the greatest value MaxbladderDepth of the maximum distance values of the respective planes is obtained.

Thereafter, the correction coefficients ComFactor[1], ComFactor[2], . . . , ComFactor[i], and ComFactor[m] for the respective planes are obtained using the greatest value MaxBladderDepth of the maximum distance values and the maximum distance values BladderDepth[1], BladderDepth[2], BladderDepth[m] of the respective planes, based on the following Equation 1.

$$ComFactor [ i ] = \frac{Max BladderDepth}{BladderDepth [ i ]} \qquad ( 1 )$$

Where ComFactor[i] is the calibration coefficient for i-th scan plane, BladderDepth[i] is the bladder depth for i-th scan plane, and MaxBladderDepth is the greatest value of the maximum distance values.

Thereafter, given the assumption that a bladder image for each plane is a circle, radii r[1], r[2], . . . , r[i], and r[m] of respective circles having the same areas as the areas Area[1], Area[2], . . . , Area[m] of the respective planes are obtained and are determined to be radii for bladder images of the respective planes at step S432.

Thereafter, calibrated radii ComR[1], ComR[2], . . . , ComR[i], and ComR[m] with respect to the correction coefficients and the calibrated radius of the bladder for each of the ultrasound scan planes is calculated using the following Equation 2:

ComR[i]=ComFactor[i]×r[i] (2)

Where, ‘ComR[i]’ is the calibrated radius of the bladder for i-th scan plane.

The average value of the calibrated radii of the bladders for the scan planes ‘AverageR’ is obtained. Thereafter, given the assumption that the complete bladder is a sphere, the total volume of urine ‘V’ in the bladder by applying the average radius ‘AverageR’ to the following Equation 3 is obtained. An average radius ‘AverageR’, which is the average value of the calculated calibrated radii for the images of the respective planes, is obtained.

$$V = \frac{4}{3} π {AverageR}^{3} \qquad ( 3 )$$

From the above-described process, the method for measuring the volume V of the bladder according to the present invention can accurately detect the amount of urine in the bladder although the transducer is placed on the position which is deviated from center of the bladder.

Although the method to measure the volume of the bladder and the amount of the urine in the bladder disclosed herein has been described for the purposes of illustration, it should be understood that various changes, modification and substitutions might be incorporated in the embodiment without departing from the spirit of the invention.

Furthermore, the apparatus according to the present invention can extract pieces of bladder information, such as the thickness and weight of the urinary bladder, as well as information about the amount of urine retaining in the urinary bladder, from two-dimensional images, and can output the pieces of extracted information of the urinary bladder to the display unit.

Although the present invention has been described in detail in conjunction with the preferred embodiment, the present invention is described only for illustrative purposes and is not limited thereto. Those skilled in the art will appreciate that various modifications and applications, which are not described above, are possible within a range that does not change the substantial characteristics of the present invention. For example, in the present embodiment, the method of obtaining an area of a bladder for a corresponding plane using the rotational angles of the first stepping motor and the second stepping motor and ultrasound information about the respective scan lines may be modified and implemented in various ways to improve scanning performance. Furthermore, it should be appreciated that the distances regarding the modifications and the applications are included in the scope of the present invention, which is defined by the accompanying claims.

What is claimed is:

1. An apparatus for measuring an amount of urine in a bladder using ultrasound signals, comprising:
a transducer for transmitting ultrasound signals and receiving ultrasound signals reflected to and from the bladder and tissues surrounding the bladder;
a switch for selecting one of operational modes, which include a bladder position check mode and a urine amount measurement mode;
a display unit for outputting images; and
a central control unit for operating the apparatus according to the operational mode selected by the switch,
wherein the central control unit in the bladder position check mode is configured to repeat sequentially predetermined operations until the urine amount measurement mode is selected, the predetermined operations include to perform a preliminary scan to obtain ultrasound signals for a plurality of scan lines of a single scan plane related to an aiming direction of the transducer, to detect location information of a bladder in a ultrasound image by using the obtained ultrasound signals of the single scan plane, to detect a center point of the bladder by using the location information of the bladder, and to represent a predetermined mark at the center point of the bladder in the displayed ultrasound image in order to indicate visually a center of the bladder, and
wherein the central control unit in the urine amount measurement mode is configured to perform an main scan to obtain ultrasound signals for a plurality of scan planes from the transducer, to estimate a volume of the bladder by using the ultrasound signals obtained by the main scan, and to measure an amount of urine corresponding to the estimated volume of the bladder.

2. The apparatus for measuring an amount of urine according to claim 1, wherein the central control unit is configured to, in the bladder position check mode, mark a vertical center line on the center of the ultrasound image.

3. The apparatus for measuring an amount of urine according to claim 1, wherein the central control unit is configured to, in the bladder position check mode, detect location information corresponding to a front and a back walls of the bladder in a plurality of ultrasound scan lines for the scan plane obtained by the preliminary scan, and extract top, bottom, right and left points of the bladder from the detected location information of the bladder in order to detect the center point of the bladder.

4. The apparatus for measuring an amount of urine according to claim 1, wherein the predetermined mark is one of a cross mark, an arrow and a geometrical figure on the center point.

5. The apparatus for measuring an amount of urine according to claim 1, wherein the central control unit is configured to, in the bladder position check mode,
calculate a cross-sectional area of the bladder in the scan plane, and determine a size of the mark representing the center point in proportion to the calculated cross-sectional area of the bladder.

6. A method for measuring an amount of urine in a bladder using ultrasound signals, comprising the steps of:
(a) providing operational modes, which include a bladder position check mode and an urine amount measurement mode;
(b) selecting the bladder position check mode and performing repeatedly and sequentially predetermined operations until the urine amount measurement mode is selected; and
(c) selecting the urine amount measurement mode, performing a main scan to obtain ultrasound signals for a plurality of scan planes from the transducer, and estimating and providing a volume of a bladder using the ultrasound signals obtained by the main scan, and
wherein the predetermined operations in the step (b) includes steps of
(b1) performing a preliminary scan to obtain ultrasound signals of a plurality of scan lines for a single scan plane from the transducer;
(b2) generating and displaying an ultrasound image of the single scan plane by using the ultrasound signals obtained by the preliminary scan;
(b3) detecting location information of a bladder in the ultrasound image by using the obtained ultrasound signals for the single scan plane;
(b4) detecting a center point of the bladder by using the location information of the bladder; and
(b5) representing a predetermined mark on the center point of the bladder in the displayed ultrasound image in order to represent a center position of the bladder.

7. The method for measuring an amount of urine according to claim 6, wherein the predetermined operations in the step (b) further includes a step of (b6) marking a vertical center line on the center of the ultrasound image.

8. The method for measuring an amount of urine according to claim 6, wherein the step of (b4) detects location information corresponding to a front and a back walls of the bladder in a plurality of ultrasound scan lines comprising the scan plan obtained by the preliminary scan, and extracts right, left, top and bottom points of the bladder from the detected location information corresponding to a front and a back walls of the bladder in order to detect the center point of the bladder.

9. The method for measuring an amount of urine according to claim 6, wherein in the step of (b5) the predetermined mark is one of a cross mark, an arrow and a geometrical figure.

10. The method for measuring an amount of urine according to claim 6, wherein the step of (b5) calculates a cross-sectional area of the bladder in the scan plane, and determines a size of the mark representing the center point in proportion to the calculated cross-sectional area of the bladder.
